(19) United States
(12) Patent Application Publication
(10) Pub. No.: US 2003/0092976 A1
(43) Pub. Date: May 15, 2003

(54) Interactive medical diagnostic device

(75) Inventors: Yasutaka Murase, Kyoto (JP); Takehiro Hamaguchi, Kyoto (JP); Takefumi Nakanishi, Kyoto (JP); Shinya Noro, Kyoto (JP); Susumu Minamikawa, Kyoto (JP)

Correspondence Address: BEYER WEAVER & THOMAS LLP, P.O. BOX 778, BERKELEY, CA, 94704-0778, US

(73) Assignee: OMRON Corporation

(21) Appl. No.: 10/288,627

(22) Filed: Nov. 4, 2002

(30) Foreign Application Priority Data
Nov. 12, 2001 (JP) 2001-345518

(51) Int. Cl. A61B 5/00

(52) U.S. Cl. 600/300

(57) ABSTRACT

An interactive medical diagnostic device includes control, display, input and memory devices. Dosage data related to administration of medicaments given to a patient and physiological values obtained by measurement from the patient are stored, and an interactive medical examination is carried out by outputting questions to and receiving answers from the patient. These data are compared and propriety of the current treatment of the patient is determined and outputted. Either data on prescribed dosage or those on actually administered dosage may be used by the control device. During the interactive medical examination, the patient may be allowed to review the earlier posed questions and supplied responses. The answers may be given not only in terms of “yes” and “no” but also as “neither.” Expected results of prescribed or actually administered dosage may be compared with the current condition obtained by measurement and/or the interactive examination and if their difference suggests that the situation is grave, a warning may be outputted to a physician in charge.

### BACKGROUND OF THE INVENTION

This invention relates to an interactive medical diagnostic device which may be used in different kinds of devices for families and medical departments for purposes such as measurements of data on a live body, health management, aids to aged and/or physically handicapped persons, and remotely controlled health data management.

There have been apparatus for asking a patient or his/her companion to fill in a questionnaire form and to present it to the receptionist of a hospital or to put it in a machine. There have also been apparatus for carrying out on a computer a portion of the medical diagnosis based on questions posed to the patient by way of a questionnaire such that the patient's replies in a multiple choice format are interactively inputted. In the past, however, it has been difficult to adjust the contents of a questionnaire so as to be applicable to different conditions of individual patients and only basic data on fundamental matters could be collected. Regarding the blood pressure, for example, it was usually a multiple-choice question such as whether or not it is within a normal range or whether it is higher or lower than a normal range. The conclusion may be only that it is higher than normal for that patient, and it was given without mentioning how high was the high pressure or what the history of the patient's blood pressure was like, and without regard to the patient's multifaceted general life style such as what are the favorite foods (whether spicy, salty or fatty) of the patient and whether he/she has been doing physical exercises regularly.

With such a questionnaire, furthermore, it is not possible to appropriately collect the effects of treatments received earlier. In the case of a high blood pressure, for example, it is not possible to distinguish whether it used to be normal but became higher recently or it dropped to the normal range because of a medical treatment but is rising again. Since the patient's physical problems could not be understood in detail from the questionnaire, the question-and-answer routine had to be repeated at the time of the physician-patient interview. This means a waste of time and a burden on both the physician and the patient.

Moreover, prior art interactive medical diagnostic systems could not correlate the details of past treatments with the contents of the questions and the answers or to appropriately manage the data obtained therefrom and the history of treatments.

### SUMMARY OF THE INVENTION

It is therefore an object of this invention in view of the problems described above to provide an apparatus capable of comparing the contents of the questions and the answers with the past history of treatments (such as dosage data), automatically investigating the contents of the interactively obtained data accordingly and more deeply, providing the physician with data which will be necessary and useful for the treatment by the physician, alleviating the burden on the physician in the management of data on the patient, and thus allowing the patient to enjoy better medical treatments.

An interactive medical diagnostic device embodying this invention, with which the above and other objects can be accomplished, may be characterized as comprising “dosage memory means” for storing dosage data, or data related to administration of medicaments for a patient inclusive of the timing of such administration of medicaments, “measurement memory means” for storing measurement data inclusive of measured physiological values obtained from the patient as well as the times at which such data (measured physiological values) were obtained, “interactive means” for carrying out an interactive medical examination by outputting questions and receiving replies to the outputted questions, and “judging means” for judging whether or not proper dosage has been being administered, based on the data stored in the aforementioned dosage memory means and the measurement memory means as well as from the results of the medical examination carried out interactively by the interactive means.

Explained more in detail, the “dosage data” include both the time at which the patient is told to take a certain medicament as well as the actual time when the patient took in the prescribed dosage. Such timing data need not be given precisely. Inputs such as “at the time of getting up” and “after breakfast” are acceptable. The measured physiological data and the times at which these measurements were taken are stored as separate data.

Propriety that is judged by the judging means may be determined in different ways. If a proper amount of a proper antihypertensive is administered to a patient with a high blood pressure, its effect will appear within a few minutes. In the case of a long-term treatment (say, with a dietary medicine), on the other hand, a number of measurements may have to be collected over an extended period of time. Expected results and the measured physiological values may have to be compared, and the difference between them and their fluctuations as well as a comparison of the difference with a specified standard value are among the processes to be carried out by the judging means. If the difference is less than such a standard value, the judgment means may conclude that an adequate dosage has been administered. If the difference is greater than the standard value, the judging means may be programmed to output a warning that the dosage should be changed. With a medical diagnostic device thus structured, situations can be avoided where a patient continues to take a medicine which is not doing any good.

The questions to be posed by the interactive means are not predetermined. Depending on the answer given to an earlier posed question, the interactive means selects the next question to be asked. This is in contrast to prior art devices programmed to ask the same set of questions in the same order without regard to the answers being given by the patient. In other words, prior art interactive means collected many useless data not pertinent to the individual patients. The interactive means according to this invention is programmed to collect emphatically only those answers that are important to the patient.

When a question is asked by the interactive means, it sometimes happens, depending on the individual characteristics of the patient or the circumstances, that the patient cannot give an immediate answer. According to a preferred embodiment of the invention, the interactive means is programmed to ignore the answer if it is not given within a specified length of time after the corresponding question was posed. It is because a patient, when confronted with a question that is difficult to answer for whatever reason, tends to feel compelled to answer independent of its veracity. Such a reply is likely to lead the physician to an erroneous conclusion. It is further preferable, if the patient hesitates before answering a question, to store this information in a memory means because a hidden personal characteristic of the patient may come to be revealed by analyzing the question regarding which the patient has hesitated in answering.

Although most questionnaires provide only two choices “yes” and “no” as a selectable reply, there can be situations where a patient cannot fairly describe his/her conditions by answering to a question in the positive or in the negative. It is helpful also to the treating physician that the patient was in a difficult position to answer “yes” or “no” to a question. For this reason, it is preferable to include “neither” as a third choice.

There are situations where the anticipated effect of the administration of a dosage is different from the actual change in the patient's condition. It may be that the administered dosage shows no or little effect. On the other hand, the situation may be that the dosage is too effective. Both are undesirable situations and a physician should take an active role in making a diagnosis. In many situations, however, such a judgment can be rendered if normal physiological data are being measured regularly. For this reason, a device according to this invention may be structured in either of the following two ways.

A device in one of these two forms may be characterized as comprising an “effect extracting means” for extracting expected effects of the dosage data stored in the aforementioned dosage memory means and storing such extracted effects and a “warning means” for outputting a warning if the expected effects stored in the effect extracting means are different from the result of the interactive medical examination carried out by the interactive means including the results of the measurement stored in the measurement memory means. A device in the other of these two forms may be characterized as comprising a “prescribed dosage memory means” for storing prescribed dosage data on a dosage prescribed to a patient and an “effect memory means” for storing expected effects of the prescribed dosage. Entries to the prescribed dosage memory means may be effected directly by the physician or the nurse in charge by means of an input device. Prescription data inputted through a separate device may be transmitted to the device of this invention to be stored in the prescribed dosage memory means.

The former is adapted to output a warning if the expected effects of the dosage actually taken in by the patient, without regard to whether or not the patient followed the physician's prescription, are determined to be different from the results of the interactive medical examination inclusive of the results of the measurement stored in the measurement memory means. The latter is adapted to output a warning if the expected effects of the dosage prescribed by the physician, without regard to what dosage was actually taken in by the patient, are determined to be different from the results of the interactive medical examination inclusive of the results of the measurement stored in the measurement memory means. With a device thus structured, the patient can be warned of an undesirable situation at an early stage.

If the expected results and the actual changes taking place in the patients are different, whether because the prescribed dosage is not effective or overly effective, the patient's life may be being threatened in the worst situation. The warning device may be arranged such that a supervisor (whether this is a physician or a nurse) in charge will be alerted if it is determined that the situation is grave.

The interactive means may be programmed so as to request a final confirmation at the end a medical examination because many patients wish to review their own answers given during the examination. This gives the patient an opportunity to correct any erroneously provided response and serves to improve the accuracy of the examination. If a patient makes a correction, this fact will be recorded such that the physician can review how changes have been made by the patient.

Since many patients want to know during the course of an interactive medical examination how they answered some of the earlier posed questions, the interactive means may be programmed to display records of earlier posed questions as well as the answers given to them, allowing the patient to make corrections at any time during the interactive medical examination. It goes without saying that any number of earlier supplied answers may be corrected.

The interactive means may be programmed to selectively allow or forbid inspection of results of past medical examination. This is to prevent uncontrolled modification of records.

### BRIEF DESCRIPTION OF THE DRAWINGS

FIG. 1 is a block diagram of a system having medical data terminals each provided with an interactive medical diagnostic device of this invention connected through the internet to a management center device.

FIG. 2 is a block diagram schematically showing the structure of a medical data terminal shown in FIG. 1.

FIG. 3 is a flowchart for showing the operation of the medical data terminal of FIG. 2.

FIG. 4 is an image which may be displayed in Step ST1 of the flowchart of FIG. 3.

FIG. 5 is an example of response which may be entered in the image shown in FIG. 4.

FIG. 6A is an image which may be displayed in Step ST2 of the flowchart of FIG. 3, and

FIG. 6B is an example of response which may be entered in the image shown in FIG. 6A.

FIG. 7 is a diagram for showing the flow of operations of the medical data terminal in Steps ST3-ST5.

FIG. 8 is an example of display in Step ST6 for a second-stage questionnaire.

FIG. 9 is another example of display in Step ST6 for the second-stage questionnaire.

FIG. 10 is an example of display in Step ST8 for a third-stage questionnaire.

FIG. 11 is an example of display in Step ST11 by the connection to the medical database.

### DETAILED DESCRIPTION OF THE INVENTION

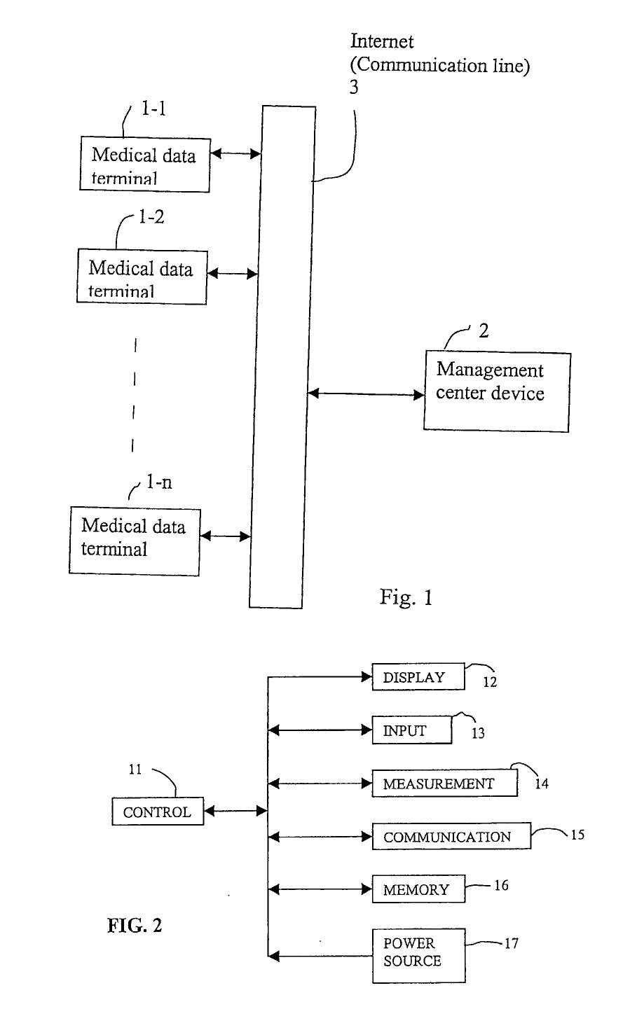

The invention is described next by way of an example of its embodiment. FIG. 1 is a block diagram of a system comprised of a plurality of medical data terminals 1-1, 1-2, . . . 1-n each provided with an interactive medical diagnostic device of this invention and connected to a management center device 2 through the internet (communication line) 3. Each of these medical data terminals 1-1, 1-2, . . . 1-n may be placed at a residential home or at a hospital reception desk, or may be carried by an individual as a portable personal data terminal. The management center device 2 may be placed at a management center of a medical organization or may be an individual terminal of a physician and is provided, for example, with a basic questionnaire database (original), a dosage administration and an individual physiological information database (master), a physiological data monitoring engine (master), a medical information database (original) and an individual medical record database (master). Each of the medical data terminals 1-1, 1-2, . . . 1-n is provided, for example, with a basic questionnaire database (only necessary portions), a dosage administration and questionnaire reasoning engine (only necessary portions), an individual physiological information database (copy), measurement devices for physiological information (such as a sphygmomanometer and a clinical thermometer), a physiological data monitoring engine (slave), a medical information database (only necessary portions), an individual medical record database (copy), data communication means and an man-to-machine interface.

As shown in FIG. 2, each of the medical data terminals 1-1, 1-2, . . . 1-n is provided with a control device 11, a display device 12, an input device 13, a measurement part 14, a communication device 15, a memory device 16 and a power source 17. The control device 11 serves to output commands to the display device 12 and the memory device 16, to receive external data from the input device 13 and the measurement part 14 and to exchange data through the internet 3 with the management center device 2 serving as an external device.

The display device 12 displays various data such as measured data (including the blood pressure and the body temperature), the contents of the questionnaire and other data. The input device 13 is for responding to questionnaires, posing questions on the use, correcting inputted data and switching displayed images. The measurement part 14 is for measuring physiological information such as the blood pressure and the body temperature. The communication device 15 is for carrying out communications with the management center device 2. The memory device 16 is for storing measured physiological data, contents of questionnaires, a questionnaire database, data on the past history of treatment on each patient, instructions from the physicians and daily records of the individual patients. The power source 17 is for supplying power for driving all these devices.

The functions of the aforementioned “dosage memory means,” “measurement memory means,” “effect memory means” and “prescribed dosage memory means” are carried out by the memory device 16. Those of the aforementioned “interactive means” are carried out by the input device 13, the display device 12 and the control device 11. Those of the aforementioned “judging means” and “warning means” are carried out by the control device 11 and the display device 12 or the communication device 15. Those of the aforementioned “effect extracting means” are carried out by the control device 11.

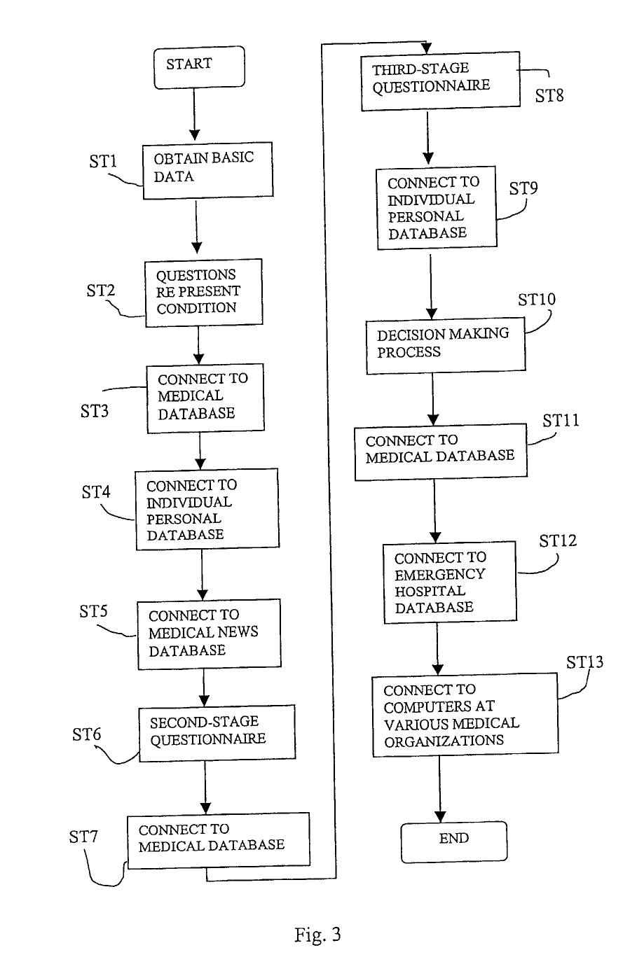

The flowchart of FIG. 3 is referenced next to explain the operations of the medical data terminals 1-1, 1-2, . . . 1-n thus structured as they exchange data with the management center device 2 through the internet 3.

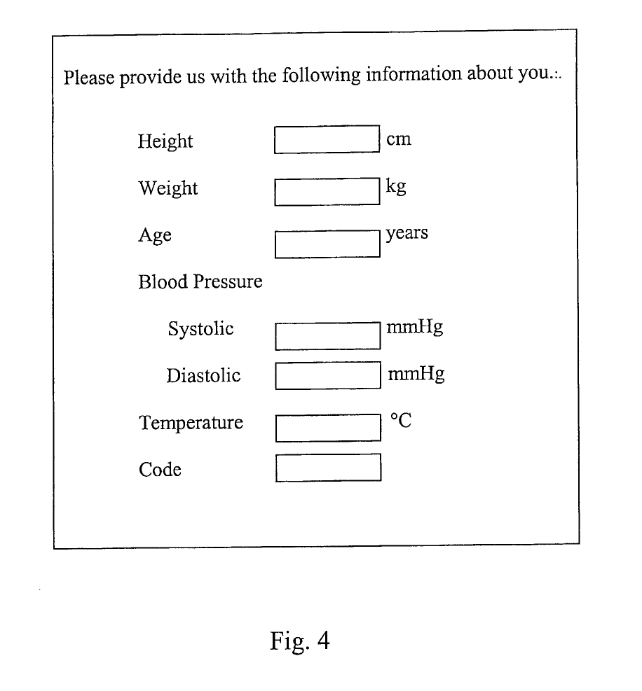

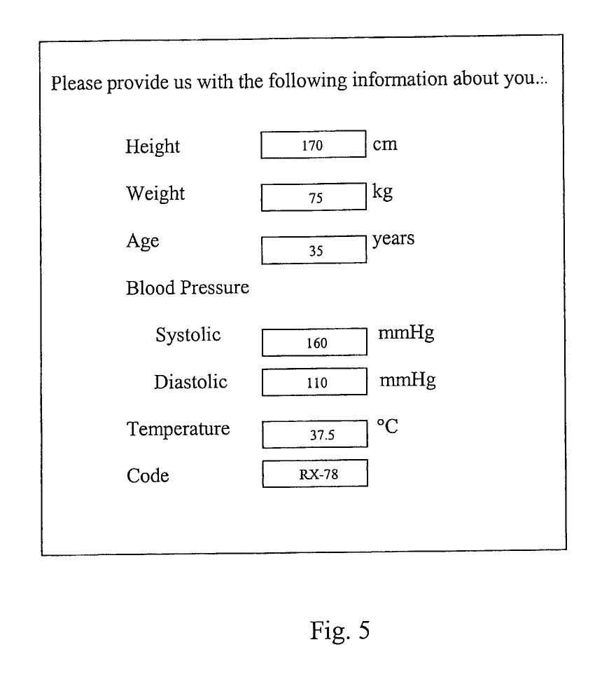

To start, basic data are obtained (Step ST1). At this moment, a display as shown in FIG. 4 is made on the display device 12, and the patient is requested to supply data on his/her body height (cm), weight (kg), age (years), systolic and diastolic values (mmHg), body temperature (° C.) and individual code. If a measurement is necessary in order to answer any of these questions, any of measuring devices of the measurement part 14 may be used. For example, the systolic and diastolic values may be obtained by using a sphygmomanometer and the body temperature may be obtained by using a clinical thermometer of the measurement part 14. FIG. 5 shows an example of replies inputted in response to the displayed questionnaire shown in FIG. 4. This is an example of a person who is somewhat heavier than the normal with respect to the height, having both the systolic and diastolic values exceeding the normal range, and the body temperature is slightly above the normal.

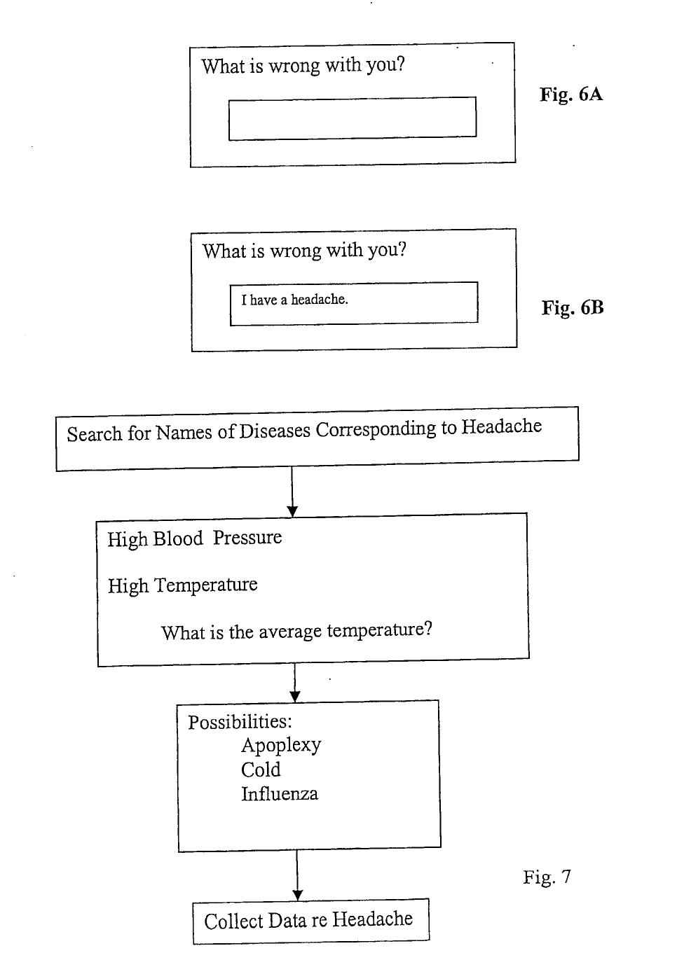

After these basic data are inputted, a question is posed as to the present health condition of the patient (Step ST2). This may be done as shown in FIG. 6A by displaying a question, to which the patient may enter a response as shown in FIG. 6B. Upon receiving this response, the device connects itself to its medical database (Step ST3), to the individual patient's personal database (Step ST4) and to the medical news database (Step ST5). Through these steps, the diagnostic device searches, as shown in FIG. 7, for the name of a disease corresponding to the headache by referencing the personal data provided as a response from the patient. In other words, on the basis of the collected information such as the high blood pressure, the high body temperature and the high average temperature, names of candidate diseases are picked up. In this example, apoplexy, cold and influenza are picked up as candidates.

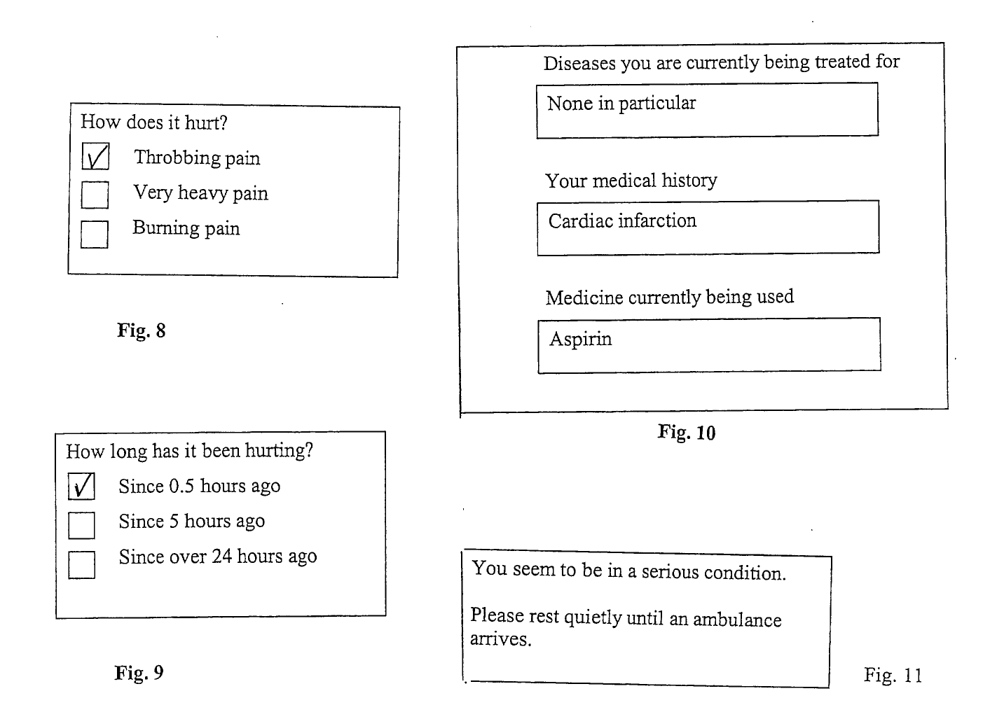

Next, a second-stage questionnaire is outputted for collecting data on headache (Step ST6). The question may be in a multiple-choice format. As the patient replies by selecting one of the choices, as shown in FIG. 8, a subsequent questionnaire is displayed. Let us suppose that the patient has responded as shown in FIG. 9. As a result, the medical database is connected again (Step ST7) to pick up names of candidate diseases that may be predictable from the patient's reply such as apoplexy and cerebral infarction. Thereafter, a third-stage questionnaire is outputted as shown in FIG. 10 (Step ST8). In this example, the patients is replying that he/she is currently not being treated for any particular disease, that his/her medical history includes cardiac infarction and that he/she is currently taking aspirin.

Upon receiving this set of replies from the patient, the medical diagnostic device is connected to the individual personal database (Step ST9) and further narrows down on the possibilities in view of these received replies. In this example, it comes to the conclusion that it is most likely a case of cerebral infarction. If it is decided in the following decision-making process (Step ST10) that the situation is grave and an emergency procedure should be followed immediately, the medical database is connected (Step ST11) to search for the step to be taken, and the recommended step to be taken is communicated to the patient. This may be a warning, for example, to rest quietly until an ambulance arrives, as shown in FIG. 11.

Next, a connection is made to an emergency hospital database (Step ST12) to search for a nearest hospital capable of handling a patient with cerebral infarction and a communication is made to the hospital of the patient and to request that an ambulance be dispatched. Still thereafter, connections are made to the computers at various medical organizations to transmit the personal data on the patient and his/her current conditions to the selected hospital and physiological data of the patient are monitored. This monitoring process is carried out by the aforementioned measurement part 14. The patient's images taken by a CCD camera, his/her body temperature measured by a temperature sensor, his/her blood pressure measured by a pressure sensor, for example, may be transmitted to the hospital.

In summary, as explained above, responses obtained interactively and the data on treatment (such as data on administered dosages) are consulted and appropriately more detailed questionnaires are outputted such that the physician can be provided with necessary and useful data when the patient is finally about to be treated. This reduces the burden on the treating physician and the patient can enjoy more effective treatments.

What is claimed is:

1. An interactive medical diagnostic device comprising:
dosage memory means for storing dosage data on a dosage for a patient including timing of administration of said dosage;
measurement memory means for storing measurement data on measurements of physiological values obtained from said patient;
interactive means for carrying out an interactive medical examination by outputting questions and receiving replies to said questions; and
judging means for judging propriety of said dosage based on said dosage data stored in said dosage memory means and said measurement data stored in said measurement memory means as well as results of said interactive medical examination.

2. The interactive medical diagnostic device of claim 1 wherein said interactive means changes the questions according to those of the replies that have been earlier received.

3. The interactive medical diagnostic device of claim 1 wherein said interactive means ignores those of the replies that are not received within a specified length of time after corresponding ones of the questions.

4. The interactive medical diagnostic device of claim 1 wherein said interactive means makes a record if said patient does not respond to any of the questions within a specified length of time.

5. The interactive medical diagnostic device of claim 1 wherein said replies include YES, NO and NEITHER.

6. The interactive medical diagnostic device of claim 1 wherein said interactive means also serves to request a final confirmation of said medical examination after said medical examination is finished.

7. The interactive medical diagnostic device of claim 1 wherein said interactive means also serves to display results of earlier examinations, correcting results of said earlier examinations and storing information that corrections on results of said earlier examinations have been made.

8. The interactive medical diagnostic device of claim 1 wherein said interactive means also serves to selectively allow or forbid inspection of results of past medical examination carried out thereby.

9. An interactive medical diagnostic device comprising:
dosage memory means for storing dosage data on a dosage administered to a patient;
effect extracting means for extracting and storing expected effects of said dosage data;
measurement memory means for storing measurement data on measurements of physiological values obtained from said patient;
interactive means for carrying out an interactive medical examination by outputting questions and receiving replies to said questions; and
a warning means for outputting a warning if said expected effects stored in the effect extracting means are determined to be different from results of said interactive medical examination carried out by the interactive means in view of said measurement data stored in said measurement memory means.

10. The interactive medical diagnostic device of claim 9 wherein said warning means serves to warn a supervisor if said dosage data and the results of said interactive medical examination indicate an abnormal condition.

11. The interactive medical diagnostic device of claim 9 wherein said interactive means also serves to request a final confirmation of said medical examination after said medical examination is finished.

12. The interactive medical diagnostic device of claim 9 wherein said interactive means also serves to display results of earlier examinations, correcting results of said earlier examinations and storing information that corrections on results of said earlier examinations have been made.

13. The interactive medical diagnostic device of claim 9 wherein said interactive means also serves to selectively allow or forbid inspection of results of past medical examination carried out thereby.

14. An interactive medical diagnostic device comprising:
prescribed dosage memory means for storing prescribed dosage data on a dosage prescribed to a patient;
effect memory means for storing expected effects of the prescribed dosage;
measurement memory means for storing measurement data on measurements of physiological values obtained from said patient;
interactive means for carrying out an interactive medical examination by outputting questions and receiving replies to said questions; and
a warning means for outputting a warning if said expected effects stored in the effect memory means are determined to be different from results of said interactive medical examination carried out by the interactive means in view of said measurement data stored in said measurement memory means.

15. The interactive medical diagnostic device of claim 14 wherein said warning means serves to warn a supervisor if said dosage data and the results of said interactive medical examination indicate an abnormal condition.

16. The interactive medical diagnostic device of claim 14 wherein said interactive means also serves to request a final confirmation of said medical examination after said medical examination is finished.

17. The interactive medical diagnostic device of claim 14 wherein said interactive means also serves to display results of earlier examinations, correcting results of said earlier examinations and storing information that corrections on results of said earlier examinations have been made.

18. The interactive medical diagnostic device of claim 14 wherein said interactive means also serves to selectively allow or forbid inspection of results of past medical examination carried out thereby.
